(19) United States
(12) Patent Application Publication
(10) Pub. No.: US 2009/0012140 A1
(43) Pub. Date: Jan. 8, 2009

(54) Preparation of Telmisartan Salts with Improved Solubility

(75) Inventors: Ljubomir Antoncic, Ljubljana (SI); Anton Copar, Smartno pri Litiji (SI)

Correspondence Address: ARENT FOX LLP, 1050 CONNECTICUT AVENUE, N.W., SUITE 400, WASHINGTON, DC, 20036, US

(73) Assignee: LEK PHARMACEUTICALS D.D, LJUBLJANA (SI)

(21) Appl. No.: 11/718,838

(22) PCT Filed: Nov. 9, 2005
(86) PCT No.: PCT/EP05/11980

§ 371 Date: Feb. 28, 2008

(30) Foreign Application Priority Data
Nov. 11, 2004 (SI) P200400309

(51) Int. Cl. A61K 31/4184; C07D 403/04; A61P 9/12

(52) U.S. Cl. 514/394; 548/458

(57) ABSTRACT

New alkali and earth-alkali salts of telmisartan in amorphous form and a new crystalline sodium salt of telmisartan have been prepared by preparing a solution despite low solubility of telmisartan and rapidly vacuum evaporating to dryness.

### FIELD OF THE INVENTION

The present invention relates to the novel salts of telmisartan and novel polymorph form thereof, to processes for their preparation and to pharmaceutical compositions containing them.

### BACKGROUND OF THE INVENTION

Telmisartan is an antihypertensive agent disclosed in EP 502314 as well as J. Med. Chem. 36 (25), 4040-4051 (1993). According to EP 1144386 it exists in crystalline modifications Form A and Form B. In J. Pharm. Sci, 89 (11), 1465-1479 (2000) polymorph Form A is characterized by m.p. 269° C. and polymorph Form B by m.p. 183° C. Additional pseudopolymorphic Form C is known. Crystallographic data on all three are given.

Different formulation approaches address the fact that telmisartan is poorly soluble in water or physiological fluids.

Telmisartan is according to WO 04028505 formulated into double layered tablets where the active pharmaceutical ingredient is after combining with a granulation liquid and a strong alkali dried by spray drying. Similarly in WO 03059327 a tablet matrix with telmisartan said to be a substantially amorphous form is disclosed. The ratio of alkali versus telmisartan in examples in solution which is being spray dried is above one.

Alternatively WO 03037876 discloses a crystalline sodium salt of telmisartan having m.p at 245±5° C.

### BRIEF DESCRIPTION OF THE DRAWINGS

FIG. 1 is an X-ray powder diffractogram of amorphous telmisartan

FIG. 2 is an IR spectra of amorphous telmisartan

FIG. 3 is an X-ray powder diffractogram of amorphous sodium salt of telmisartan

FIG. 4 is an IR spectra of amorphous sodium salt of telmisartan

FIG. 5 is an X-ray powder diffractogram of amorphous potassium salt of telmisartan

FIG. 6 is an IR spectra of amorphous potassium salt of telmisartan

FIG. 7 is an X-ray powder diffractogram of amorphous magnesium salt of telmisartan

FIG. 8 is an IR spectra of amorphous magnesium salt of telmisartan

FIG. 9 is an X-ray powder diffractogram of amorphous calcium salt of telmisartan

FIG. 10 is an IR spectra of amorphous calcium salt of telmisartan

FIG. 11 is an X-ray powder diffractogram of Form 2 sodium salt of telmisartan

FIG. 12 is an IR spectra of sodium salt of telmisartan Form 2

FIG. 13 is an DSC thermogram of Form 2 sodium salt of telmisartan

FIG. 14 is a comparison of DSC thermograms of various salts and forms of telmisartan

### DISCLOSURE OF THE INVENTION

In general aspect our intentions are new alkali and earth alkali salts of telmisartan, as well as amorphous telmisartan which have substantially more soluble in water than Forms A or B.

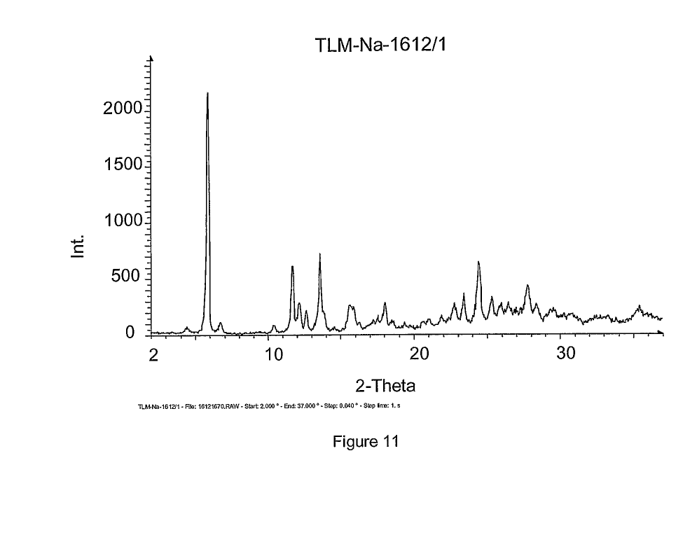

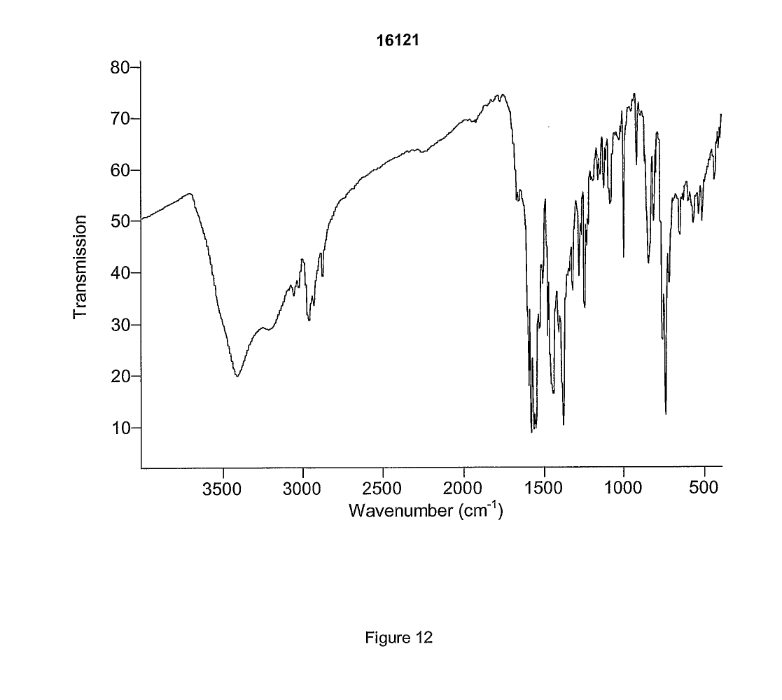

One aspect of the inventions is thus a crystalline sodium salt of telmisartan with an X-ray powder diffraction pattern exhibiting strongest diffractions at 5.8; 11.6; 13.5; 24; 4±0.2° 2Theta and preferably additionally exhibiting diffractions at 12.1; 15.6; 15.9; 18.0; 22.7; 23.4; 25.3; 25.9; 26.4; 27.0; 27.8; 28.4; 29.3; 35.4±0.2° 2Theta or essentially as on FIG. 11 or IR spectra essentially as on FIG. 12.

Other aspects of the invention are a potassium salt of telmisartan; (m.p 183-188.2° C.); a magnesium salt of telmisartan (m.p.: 216-230° C.); a calcium salt of telmisartan (m.p. 208-214° C.); and in another aspect amorphous sodium salt having m.p. around 195° C., (preferably broad m.p. 185-205° C.); and in yet another aspect amorphous telmisartan, characterized by an X-ray powder diffraction pattern exhibiting a continuum of diffractions substantially throughout the measured range from 20 to 37° 2Theta.

Specific aspects of the invention are telmisartan and/or its salts exhibiting solubility above 10 μg/ml, preferably above 50 μg/ml, more preferably above 5 mg/ml, yet more preferably above 50 mg/ml in phosphate buffer at pH 6.8 after stirring 50 mg for 30 minutes at 37° C. in 100 ml baker at 600 rpm and another aspect is the process to prepare them, characterized in that it comprises the steps of
providing a solution of telmisartan or its salt in a solvent selected from group consisting of water, alcohol, chlorinated solvent and alkane; and removing the solvent.

The solubility aspect of telmisartan and/or the salts of the invention is characterized in that telmisartan or its salt exhibit solubility above 50 μg/ml, preferably above 500 μg/ml, more preferably above 5 mg/ml or 100 mg/ml in phosphate buffer at pH 6.76, additionally having sodium taurocholate in concentration 2.5 mM and lecitin in concentration 0.5 mM after stirring 50 mg for 30 minutes at 37° C. in 100 ml baker at 600 rpm.

Aspects of the invention are processes:

for preparing amorphous alkali or earth alkali salts of telmisartan which comprises steps:
adding a solvent selected from group consisting of water, alcohol, chlorinated solvent and alkane in a five to fiftyfold excess relative to the mass of solute to form a suspension of telmisartan;
contacting suspension obtained in step a) with at least equimolar quantity of an alkali or earth alkali alcoholate or hydroxide to form a solution of an alkali or earth alkali salt of telmisartan;
optionally filtering; and
vacuum evaporating to dryness or lyophilizing the obtained solution,
   or
   for preparing amorphous telmisartan which comprises steps:
dissolving telmisartan in a chlorinated solvent or in tetrahydrofuran in a ten to fiftyfold excess relative to the mass of solute to form a clear solution;
evaporating solution obtained in step a) to dryness in vacuum and temperature 40-60° C.
   or for preparing crystalline sodium salt of telmisartan Form 2 which comprises steps:
suspending telmisartan in toluene at room temperature;
reacting suspension obtained in above step with dissolved in mixture of ethanol and water at elevated temperatures;
filtering said reaction mixture and stirring clear filtrate at lower temperature; and
isolating sodium salt formed in above step.

Within the scope of the invention are contemplated the use of either amorphous telmisartan and/or of amorphous sodium or magnesium or potassium or calcium salt of telmisartan and/or of a crystalline sodium salt of telmisartan characterized by an X-ray powder diffraction pattern exhibiting characteristic diffractions at about 5.8; 11.6; 13.5; 24; 4±0.2° 2Theta as a medicament.

The additional scopes of the invention are a pharmaceutical composition comprising amorphous telmisartan produced or sodium salt of telmisartan characterized by an X-ray powder diffraction pattern exhibiting characteristic diffractions at about 5.8; 11.6; 13.5; 24; 4±0.2° 2Theta and a pharmaceutically acceptable carrier.

### DESCRIPTION OF THE INVENTION

Present invention discloses new salts of telmisartan and novel amorphous form of telmisartan. A sodium salt has been prepared in new crystalline modification which we named Form 2. The object of the present invention are also processes for their preparation and pharmaceutical compositions containing them. The substances in accordance with our invention provide advantageous dissolution properties.

In case telmisartan, which may be obtained by the known methods, is dissolved in a solvent, preferably in chlorinated or etheric solvent, most preferably in dichloromethane or chloroform or THF in and the obtained solution is vacuum evaporated to dryness, an amorphous solid is formed, consisting only of telmisartan, without any additives, that exhibits a continuum of X-ray diffractions throughout the entire diffractogram scale. The obtained amorphous form is galenicaly advantageous, since it dissolves more rapidly than the known crystalline forms.

Analogously telmisartan dissolved in suitable solvent can be converted by an alkali or earth alkali hydroxide or alkoxide to the respective alkali or earth alkali salt and vacuum evaporation of a solution to dryness produces amorphous solids characterized in that they exhibit a continuum of X-ray diffractions throughout the entire diffractogram scale. Surprisingly it is possible to produce a novel crystalline sodium salt of telmisartan, which exhibits in an X-ray powder diffraction diagram the most characteristic peaks at about 5.8; 11.6; 13.5; 24; 4±0.2° 2Theta, and advantageous dissolution properties.

The processes for preparing amorphous telmisartan or its alkali or earth alkali salts share the common feature of preparing a solution despite low solubility and rapidly vacuum evaporated to dryness. In accordance with the scope of our invention the work up can be performed in laboratory scale batches from 0.5 g to 12 g of telmisartan in suitable vessels of 0.2 to 2 liters by evaporating in vacuum with pressure bellow 5 mbars (final vacuum) at higher temperatures, preferably at above 50° C., Evaporation can be perfumed very fast so that approximately 25-80 ml of solvent per minute is evaporated if the batch size is approximately 10-100 g. the evaporation proceeds at high temperatures, in one embodiment above 50° C., preferably above 60° C. or at temperature ranges temperatures of 30° C. to 80° C. or preferably 40-60° C. Alternatively in industrial scale batches in vessels of suitable size the processes can be devised to run continuously.

In accordance with the present invention, there are provided pharmaceutical composition comprising amorphous telmisartan or sodium or magnesium or calcium or potassium salt of telmisartan alone or in combination with another active ingredient such as hydrochlorotiazide and a pharmaceutically acceptable carrier comprising inactive ingredients such as fillers (diluents), binders, disintegrants, glidants, lubricants and other excipients.

Pharmaceutical compositions can be in a form suitable for peroral or parental application. Pharmaceutical composition in accordance with this invention can be embodied for example in form of tablet, capsules, pellets, granules and suppositories or their combined forms. Solid pharmaceutical compositions can be shielded, for example coated with the aim of increasing peletibility or regulating the disintegration or absorption.

### DETAILED DESCRIPTION OF THE INVENTION

### A New Amorphous Form of Telmisartan

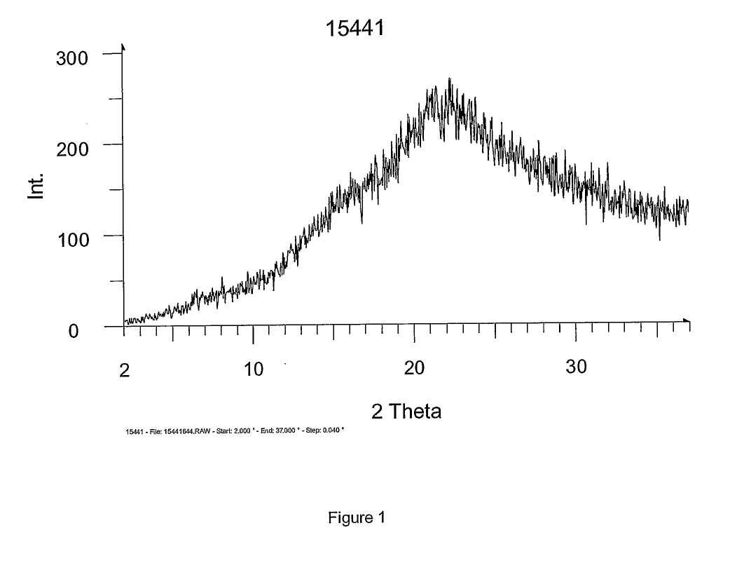

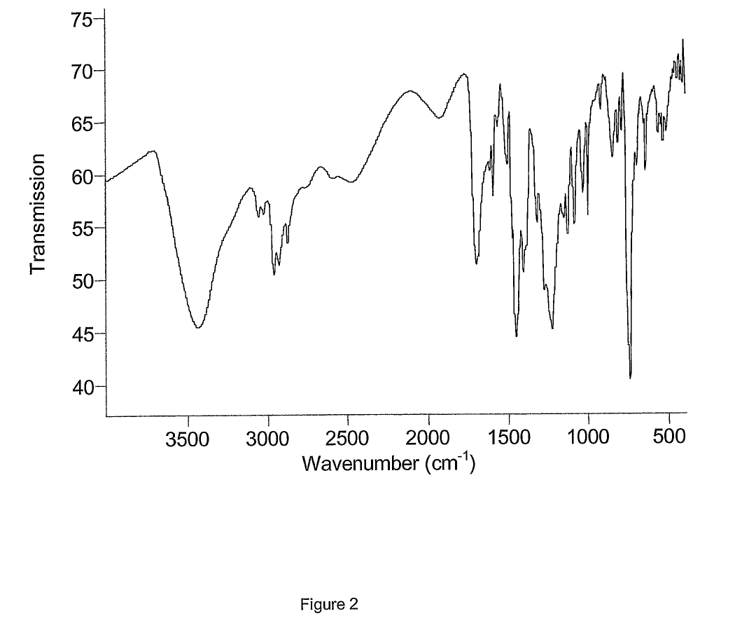

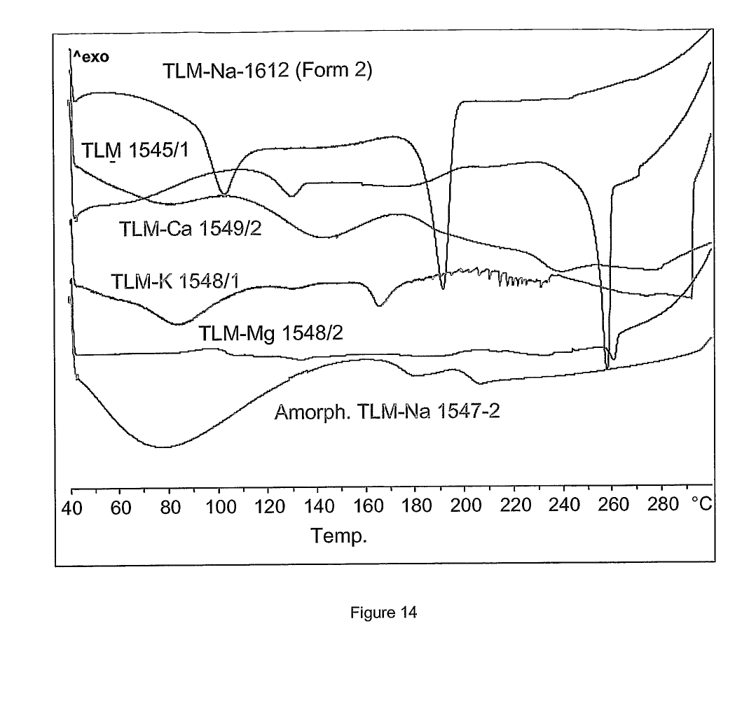

When telmisartan, which may be obtained by the known methods, is dissolved in chlorinated or etheric solvent, for example in dichloromethane or chloroform or THF in amount of 0.02 g/ml-0.2 g/ml at room temperature and the obtained solution is filtered and vacuum evaporated to dryness, an amorphous solid is formed characterized by an X-ray powder diffraction which does not exhibit any significant peaks and is further characterized by fairly broad range of m.p. around 150° C.; DSC as on FIG. 14; IR as on FIG. 2. FIG. 1 shows typical X-ray powder diffractogram of amorphous telmisartan

### Novel Salts of Telmisartan

Telmisartan will normally not produce a solution, but a suspension even in an excess (such as fivefold or tenfold or even fiftyfold relative to the mass of solute) of solvent, such as water or an alcohol such as methanol, ethanol or n-propanol or i-propanol or chlorinated solvent such as dichloromethane or an alkane such as methycyclohexane. However contacting such suspension with at least equimolar quantity of an alkaline source of alkali or earth alkali atoms, and allowing the reaction to proceed, will produce a solution of an alkali or earth alkali salt of telmisartan. Optionally filtering and vacuum evaporating to dryness will produce an amorphous solid. Amorphous sodium salt can be produced from the above described solution also by lyophilization.

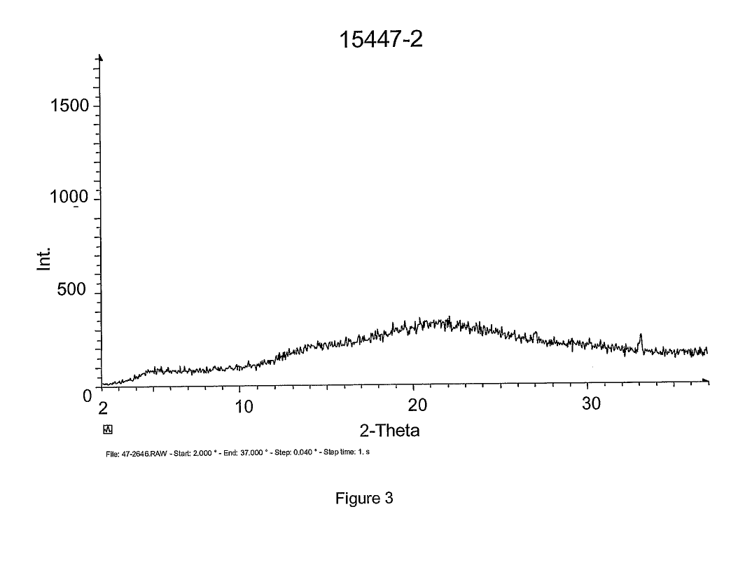

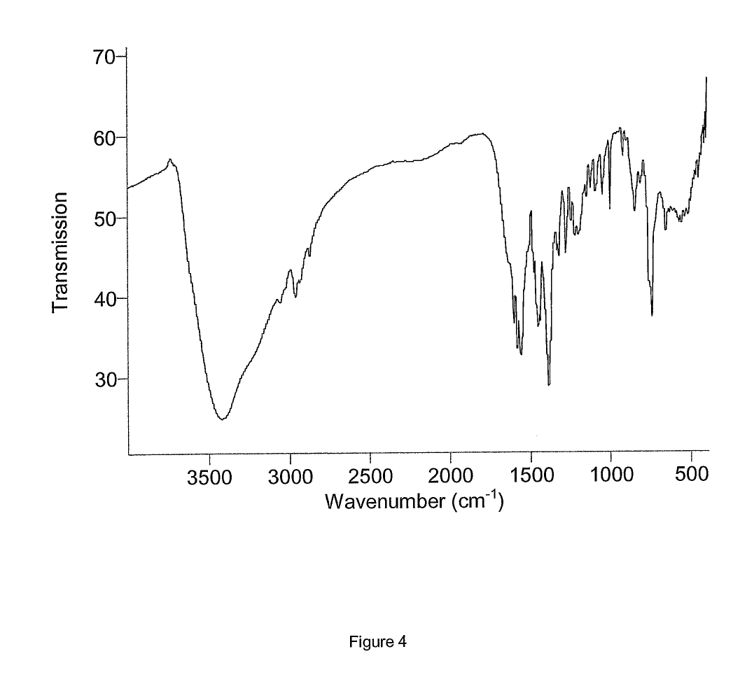

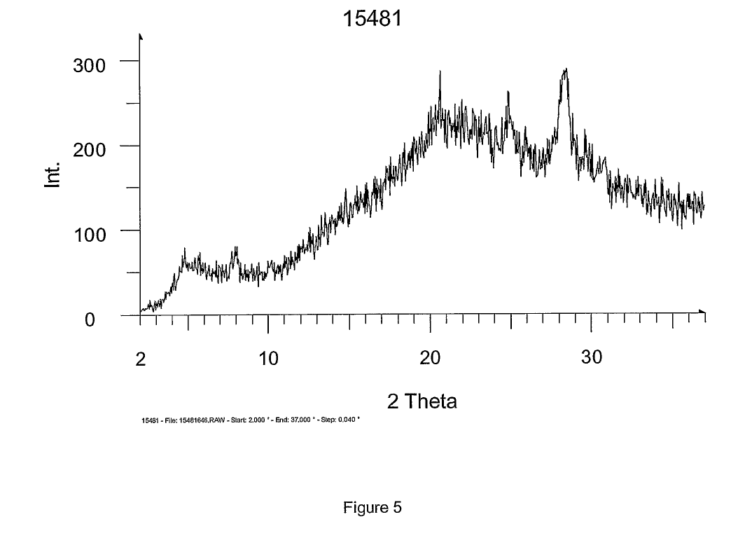

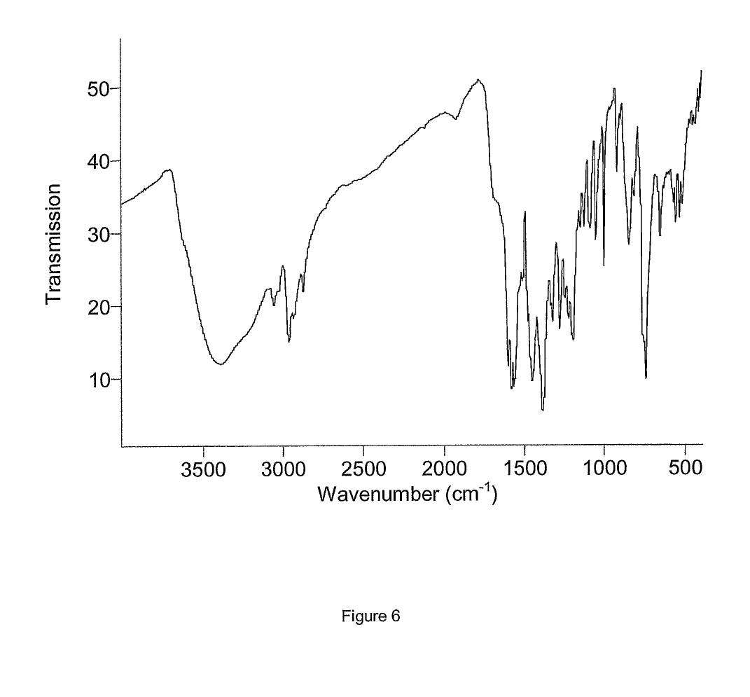

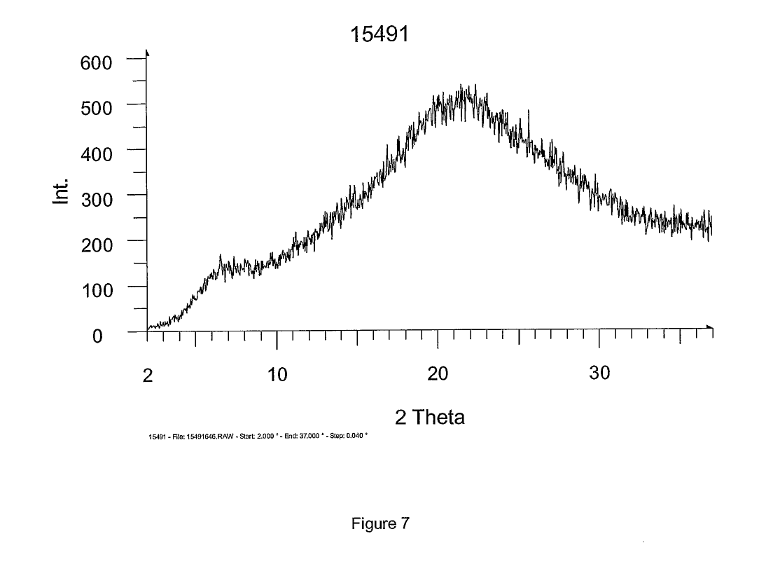

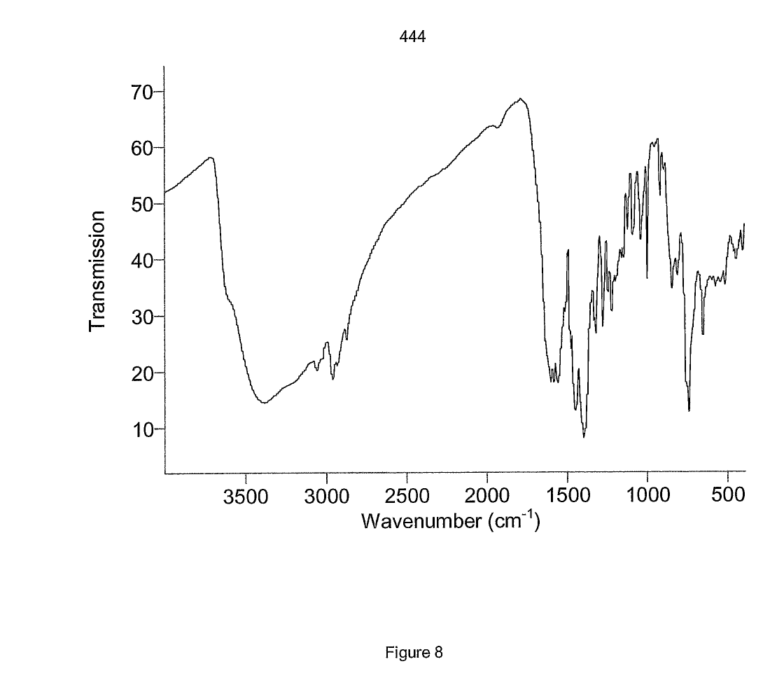

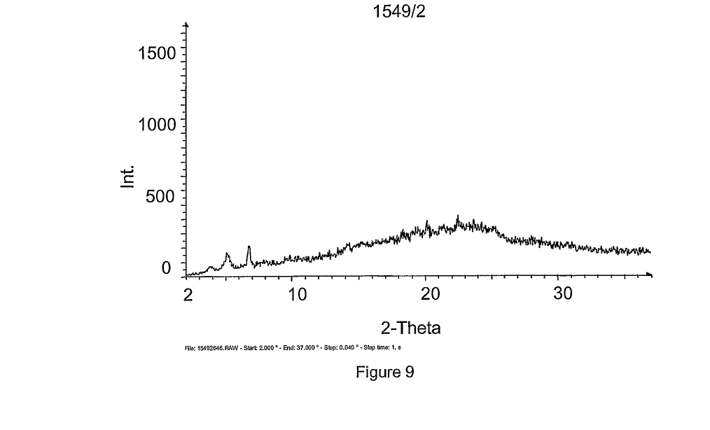

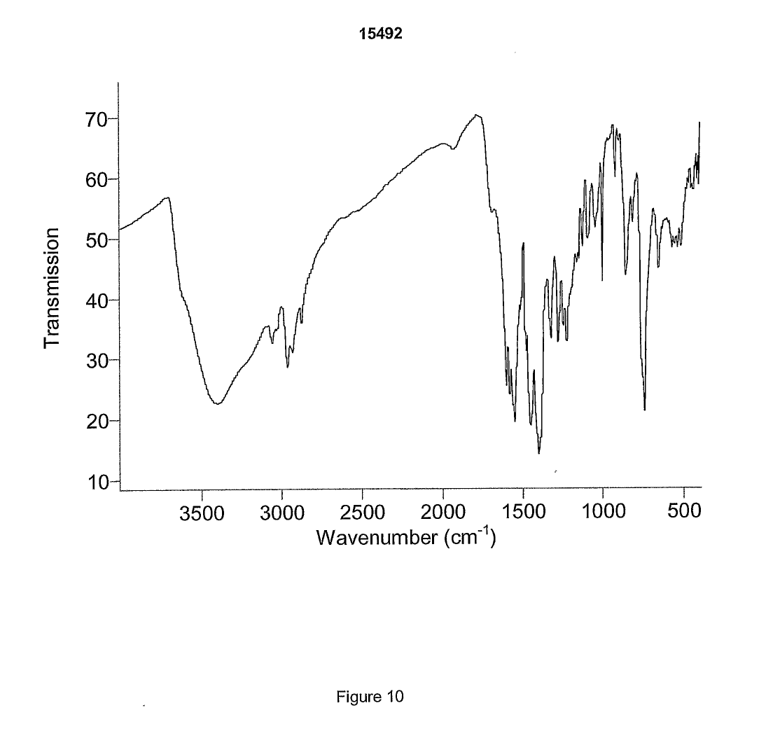

Each if the respective amorphous alkali or earth alkali salts is characterized by an X-ray powder diffraction which does not exhibit any significant peaks and respective salts are further characterized by m.p as listed in examples. Their representative X-ray powder diffractograms and IR spectra are presented on FIGS. 3 to 10, as well as DSC on FIG. 14.

### Novel Crystalline Sodium Salt of Telmisartan

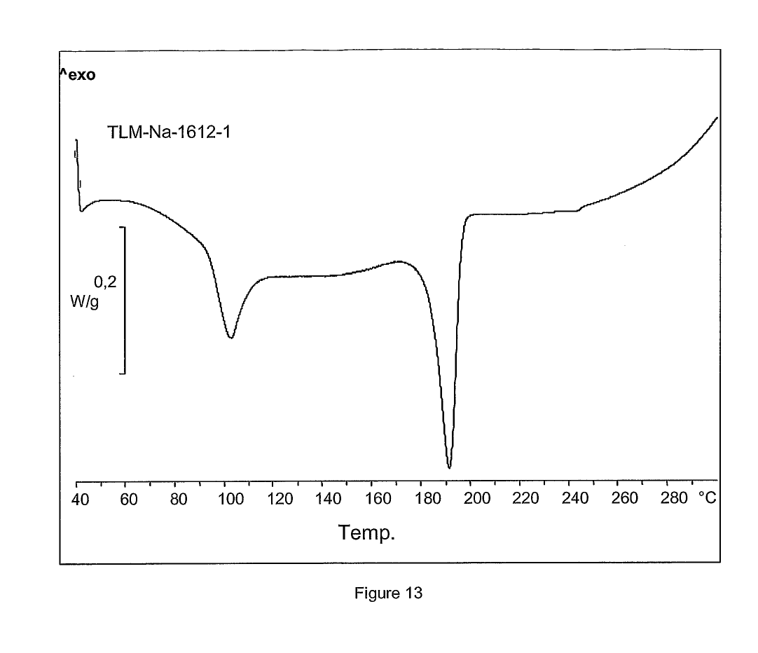

Novel crystalline sodium salt of telmisartan can be prepared by reacting telmisartan with NaOH/ethanol in toluene at elevated temperatures, filtering reaction mixture and stirring filtrate at lower temperature, whereupon solid crystallizes in a Form 2. The novel crystalline form is characterized by an X-ray diffraction pattern presented on FIG. 11, m.p around 200° C. (198.2° C.-203° C.), DSC substantially as shown on FIG. 13 and IR spectra substantially as shown on FIG. 12. The novel crystalline sodium salt of telmisartan is for example characterized by an X-ray powder diffraction pattern comprising peaks selected from peaks at about 5.8; 11.6; 12.1; 13.5; 15.6; 15.9; 18.0; 22.7; 23.4; 24.4; 25.3; 25.9; 26.4; 27.0; 27.8; 28.4; 29.3; 35.4±0.2° 2Theta. Of those the most characteristic are the peaks at about 5.8; 11.6; 13.5; 24.4±0.2° 2Theta; those peaks will normally be also the strongest. Elevated temperature will be preferably above room temperature, more preferably above 40° C., most preferably around 80° C. or above; on the other hand lower temperature will be room temperature or lower, more preferably below 20° C.

It is common feature of all those salts as well as of the amorphous telmisartan that they have better dissolution properties than known telmisartan.

Prepared novel forms of telmisartan can be incorporated into pharmaceutical formulations, which can be the solid dosage forms, for example tablets. Tablets can be prepared by methods described in WO 03059327 or WO 04028505. However the improved solubility properties allow the preparation of the solid dosage forms by conventional method. Tablet can be for example manufactured by direct compression though wet granulation is another commonly used technique. In wet granulation at least one of the ingredients can be mixed or contacted with liquid and further processed to provide aggregates, the liquid can be partially or completely removed and optionally other or more of the same ingredients may be further added and solid dosage forms manufactured.

Tableting compositions may have in addition to active pharmaceutical ingredient few or many components depending upon the tableting method used, the release rate desired and other factors. For example, compositions of the present invention may contain inactive ingredients (excipients) which function as such as different fillers, binders, disintegrants, glidants, lubricants and excipients that enhance the absorption of drugs from gastrointestinal tract.

Suitable fillers may be selected from microcrystalline cellulose, powdered cellulose, lactose, starch, pregelatinized starch, sucrose, glucose, mannitol, sorbitol, calcium phosphate, calcium hydrogen phosphate, aluminium silicate, sodium chloride, potassium chloride, calcium carbonate, calcium sulphate, dextrates, dextrin, maltodextrin, glycerol palmitostearate, hydrogenated vegetable oil, kaolin, magnesium carbonate, magnesium oxide, polymethacrylates, talc, and others. Preferred fillers are microcrystalline cellulose and lactose. Suitable binders may be starch, pregelatinized starch, gelatine, sodium carboxymethylcellulose, polyvinylpyrrolidone, alginic acid, sodium alginate, acacia, carbomer, dextrin, ethylcellulose, guar gum, hydrogenated vegetable oil, methylcellulose, hydroxyethylcellulose, hydroxypropylcellulose, hydroxypropylmethylcellulose, glucose syrup, magnesium aluminium silicate, maltodextrin, polymethacrylates, zein. Preferably hydroxypropyl cellulose, hydroxypropyl methylcellulose and polyvinylpyrrolidone are used. Suitable disintegrants may be selected from starch, pregelatinized starch, sodium starch glycolate, sodium carboxymethylcellulose, cross-linked sodium carboxymethylcellulose, calcium carboxymethylcellulose, methylcellulose, microcrystalline cellulose, powdered cellulose, polacrilin potassium, cross-linked polyvinylpyrrolidone, alginic acid, sodium alginate, colloidal silicon dioxide, guar gum, magnesium aluminium silicate, and others. Preferred disintegrants are sodium starch glycolate, cross-linked carboxymethylcellulose sodium and cross-linked polyvinylpyrrolidone. Suitable glidants may be magnesium stearate, calcium stearate, aluminium stearate, stearic acid, palmitic acid, cetanol, stearol, polyethylene glycols of different molecular weights, magnesium trisilicate, calcium phosphate, colloidal silicon dioxide, talc, powdered cellulose, starch and others. Preferred glidant is colloidal silicon dioxide. Suitable lubricants may be selected from stearic acid, calcium, magnesium, zinc or aluminium stearate, siliconized talc, glycerol monostearate, glycerol palmitostearate, hydrogenated castor oil, hydrogenated vegetable oil, mineral oil, light mineral oil, polyethylene glycol, sodium benzoate, sodium lauryl sulphate, sodium stearyl fumarate, talc and others. Preferred lubricants are calcium or magnesium stearate and stearic acid. Suitable absorption enhancers may be selected from surface active agents, fatty acids, middle chain glycerides, steroide detergents (salts of bile salts), acyl carnitine and alcanoloil choline (esters of carnitine and choline and fatty acids with middle chain and long chain), N-acyl derivatives of alpha-amino acids and N-acyl derivatives of non-alpha-amino acids, chitosanes and other mucoadhesive polymers. Especially suitable absorption enhancers are sodium deoxycholate, sodium taurocholate, polisorbate 80, sodium lauryl sulfate, sodium dodecylsulfate, octanoic acid, sodium docusate, sodium laurate, glyceride monolaurate, stearic acid, palmitinic acid, palmitooleinic acid, glycerilmonooleate, sodium taurocholate, ethylenediaminetetraacetic acid, sodium edentate, sodium citrate, b-cyclodextrine and sodium salicylate.

Different salts or esters and different polymorph forms require different techniques Pharmaceutical composition comprising novel forms of amphoteric telmisartan or its salts incorporated into a suitable pharmaceutically acceptable carrier, which may comprise above excipients can be prepared by suitable procedures for example by dry granulation or peletization. In one embodiment of the invention one can prepare tablets by direct compression. The pharmaceutical compositions can be optionally subsequently film coated.

### EXPERIMENTAL PART

Infrared spectra were obtained with Nicolet Nexus FTIR spectrophotometer. Samples were analyzed in KBr and scanned from 400 to 4000 cm⁻¹ with 16 scans and 2 cm⁻¹ resolution.

Thermograms were obtained with Mettler Toledo DSC822e differential scanning calorimeter. The sample (4-6 mg) was placed in an unsealed aluminium pan with one hole and heated at 5° C./min in the temperature range from 30° C. to 200° C. in the nitrogen (100 ml/min).

Powder X-ray diffraction spectra of the samples were recorded on Philips PW1710 with reflexion technique: CuKα radiation, range from 2° to 37° 2Theta, step 0.04° 2Theta, integration time 1 sec.

From an X-ray diffraction pattern of a powdery substance one can establish differences among different crystal lattices, and can obtain information on level of order i.e. crystallinity where lover crystallinity causes peaks to broaden. The ultimate form of non orderness of a solid is amorphous state which does not show the repeatability of molecular directions and positions in a solid. Completely amorphous substance thus shows diffuse dispersion of a X-ray radiation, which exhibits a continuum of diffractions throughout of whole the measured range. The diffraction values for a crystalline substance will be substantially independent of the diffractometer used, if the diffractometer is calibrated the values can differ for about 0.05° 2 Theta, taking into account the rounding the differences in values lay in the order of ±0.1° 2Theta, however the different recording conditions or differences in preparing or handling samples can cause the variations from the values reported for as much as ±0.2 2Theta. The intensities of each specific diffraction peak are a function of various factors, one of those being a particle size and preferred orientation. Skilled person will recognize the form from the whole X-ray powder diffraction patterns and specifically from the strongest peaks or any three to five or more distinct peaks selected from the listed peaks.

In order to prepare a pharmaceutical composition physical properties were measured. After 30 minutes at 37° C. in 100 ml baker (50 mg of compound while stirring at 600/min) the amount as presented in following table did dissolve. The solubility of the telmisartan of its salt in accordance with our invention surpasses the solubility of known forms of telmisartan, and are above 10 μg/ml in phosphate buffer pH=6.8 and above 50 μg/ml in a physiologically relevant medium if measured as above. The solubility of sodium salt of telmisartan manufactured by spray drying substantially surpasses that manufactured by lyophilization. Also the solubility of crystalline sodium salt of telmisartan on our invention surpasses that of the sodium salt of telmisartan manufactured by lyophilization. The salts are also very soluble in water (i.e dissolving approximately 500 mg/mL of Na salt Form 2 produces a very viscous solution, which can hardly be stirred with a magnetic stirrer.

| Sample/medium | | C_{30 min} (μg/ml) |
|---|---|---|
| Form A | pH = 1.2 | >500 |
| | pH = 6.8 | 0.9 |
| | (Physiologically relevant | 1.1 |
| | medium) | |
| Amorphous telmisartan | pH = 1.2 | NA |
| | pH = 6.8 | 17.6 |
| | (Physiologically relevant | 87.5 |
| | medium) | |
| Amorphous Na salt of | pH = 1.2 | 394.6 |
| telmisartan | pH = 6.8 | 29 |
| | (Physiologically relevant | 167.3 |
| | medium) | |

Physiologically relevant medium is a phosphate buffer at pH '2 6.76 + Sodium taurocholate (2.5 mM) + Lecitin (0.5 mM).

Following examples further illustrate the invention, They are provided for illustrative purposes only and are not intended to limit in any way the invention.

### Experiment 1 Na Salt of Telmisartan (Batch 1547/1.2)

2.573 g of telmisartan was suspended in 25 ml of dichloromethane (alternatively: methylcyclohexane, methanol, i-propanole). With stirring at room temperature 0.27 g sodium methoxide was added. Clear solution was filtered and vacuum evaporated to dryness. Yield: 2.33 g m.p.: 187.5° C.-200° C.

### (Batch 15472)

2.573 g of telmisartan was suspended in 25 ml of demineralised water. With stirring at room temperature 0.2 g of NaOH was added, clear solution was filtered and lyophilised. Yield: 2.47 g m.p.: 196.2° C.-202° C.

### Experiment 2 Amorphous Ca Salt of Telmisartan (Batch 15492)

2.573 g of telmisartan was suspended in 125 ml of methanol. With stirring 0.185 g of calcium hydroxide was added and stirring was continued at the temperature of reflux overnight. Reaction mixture was filtered and vacuum evaporated to dryness. Yield: 2.68 g m.p.: 208° C.-214° C.

### Experiment 3 Amorphous Mg Salt of Telmisartan (Batch 15491)

To a suspension of 2.573 g of telmisartan in 125 ml of methanol 0.29 g of Mg ethoxide was added. Reaction mixture was stirred at the temperature of reflux overnight. Reaction mixture was filtered and vacuum evaporated to dryness. Yield: 2.81 g m.p.: 216° C.-230° C.

### Experiment 4 Amorphous K Salt of Telmisartan (Batch 15481)

To a stirred suspension of 2.573 g of telmisartan in 125 ml of dichloromethane 0.561 g of K t-butoxide was added. Stirring was continued at room temperature for one hour, filtered and vacuum evaporated to dryness. Yield: 2.97 g m.p.: 183° C.-188.2° C.

### Experiment 5 Amorphous Telmisartan (Batch 15442)

A solution of 0.5 g of telmisartan in 50 ml of dichloromethane was filtered and vacuum evaporated to dryness. Yield: 0.5 g m.p.: 156° C.-161° C.

### (Batch 15441)

A solution of 0.5 g of telmisartan in 50 ml of chloroform was filtered and vacuum evaporated to dryness. Yield: 0.5 g m.p.: 155.6° C.-159.2° C.

### (Batch 15452)

A solution of 0.5 g of telmisartan in 50 ml of THF was filtered and vacuum evaporated to dryness. Yield: 0.5 g m.p.: 142.6° C.-156.2° C.

### Experiment 6 Crystalline Na Salt of Telmisartane Form 2 (Batch 16121)

15.44 g of telmisartan was suspended in 30 ml of toluene at room temperature. Suspension was reacted with mixture of 2.78 g of NaOH 44.68% (water solution) and 8.49 ml of ethanol at 80° C. Reaction mixture was stirred at the same temperature for 1 h 30 min and filtered. Clear filtrate was stirred at room temperature until white suspension was obtained, filtered and vacuum dried at 50° C. Yield: 4.64 g Form 2 m.p.: 198.2° C.-203° C.

1. A crystalline sodium salt of telmisartan characterized by an X-ray powder diffraction pattern exhibiting strongest diffractions at 5.8; 11.6; 13.5; 24; 4±0.2° 2Theta.

2. A crystalline sodium salt of telmisartan according to claim 1 additionally characterized by an X-ray powder diffraction pattern additionally exhibiting diffractions at 12.1; 15.6; 15.9; 18.0; 22.7; 23.4; 25.3; 25.9; 26.4; 27.0; 27.8; 28.4; 29.3; 35.4±0.2° 2Theta.

3. A crystalline sodium salt of telmisartan characterized by an X-ray powder diffraction pattern exhibiting characteristic essentially as on FIG. 11.

4. A crystalline sodium salt of telmisartan having IR spectra essentially as on FIG.

5. The crystalline sodium salt of telmisartan according to claim 1 characterized by melting point in range 198.2-203° C.

6. The crystalline sodium salt of telmisartan according to claim 1, which is a potassium salt of telmisartan.

7. Potassium salt of telmisartan according to claim 6 characterized by melting point in range 183-188.2° C.

8. The crystalline sodium salt of telmisartan according to claim 1, which is a magnesium salt of telmisartan.

9. Magnesium salt of telmisartan according to claim 8 characterized by melting point in range 216-230° C.

10. The crystalline sodium salt of telmisartan according to claim 1, which is a calcium salt of telmisartan.

11. Calcium salt of telmisartan according to claim 10 characterized by melting point in range 208-214° C.

12. (canceled)

13. Process for preparing telmisartan or its salt having solubility above 10 μg/ml in phosphate buffer at pH 6.8 after stirring 50 mg for 30 minutes at 37° C. in 100 ml baker at 600 rpm characterized in that it comprises the steps of
a) providing a solution of telmisartan or its salt in a solvent selected from group consisting of water, alcohol, chlorinated solvent and alkane; and
b) removing the solvent.

14. Process according to claim 13 further characterized in that telmisartan or its salt exhibit solubility above 50 μg/ml in phosphate buffer at pH 6.76, additionally having sodium taurocholate in concentration 2.5 mM and lecitin in concentration 0.5 mM after stirring 50 mg for 30 minutes at 37° C. in 100 ml baker at 600 rpm.

15. Process according to claim 13 where prepared telmisartan or it's salt is amorphous.

16. Process for preparing amorphous alkali or earth alkali salts of telmisartan which comprises steps:
a) adding a solvent selected from group consisting of water, alcohol, chlorinated solvent and alkane in a five to fiftyfold excess relative to the mass of solute to form a suspension of telmisartan;
b) contacting suspension obtained in step a) with at least equimolar quantity of an alkali or earth alkali alcoholate or hydroxide to form a solution of an alkali or earth alkali salt of telmisartan;
c) optionally filtering; and
d) vacuum evaporating to dryness or lyophilizing the obtained solution.

17. Process for preparing amorphous telmisartan which comprises steps:
a) dissolving telmisartan in a chlorinated solvent or in tetrahydrofuran in a ten to fiftyfold excess relative to the mass of solute to form a clear solution;
b) evaporating solution obtained in step a) to dryness in vacuum and temperature 40-60° C.

18. Process for preparing crystalline sodium salt of telmisartan Form 2 which comprises steps:
a) suspending telmisartan in toluene at room temperature;
b) reacting suspension obtained in step a) with dissolved in mixture of ethanol and water at elevated temperatures;
c) filtering said reaction mixture and stirring clear filtrate at lower temperature; and
d) isolating sodium salt formed in step c).

19. Process according to claim 18 where elevated temperature is above 40° and lower temperature is room temperature or lower.

20. (canceled)

21. (canceled)

22. (canceled)

23. (canceled)

24. A pharmaceutical composition comprising amorphous telmisartan produced according to claim 18 and a pharmaceutically acceptable carrier.

25. A pharmaceutical composition comprising sodium salt of telmisartan according to claim 1 and a pharmaceutically acceptable carrier.

26. Method of treating hypertension by administering a sodium salt of telmisartan according to claim 1 to a patient in need thereof.
